(19) United States
(12) Patent Application Publication
(10) Pub. No.: US 2014/0335523 A1
(43) Pub. Date: Nov. 13, 2014

(54) GENDER-SPECIFIC IDENTIFICATION OF SPERM CELLS AND EMBRYOS USING LOCKED NUCLEIC ACIDS

(71) Applicant: MofA Group LLC, Shawano, WI (US)

(72) Inventors: Bradley Didion, Shawano, WI (US); John Verstegen, Shawano, WI (US); Patrick Hrdlicka, Shawano, WI (US)

(21) Appl. No.: 14/369,949

(22) PCT Filed: Jan. 3, 2013
(86) PCT No.: PCT/US13/20139

§ 371 Date: Jun. 30, 2014

Related U.S. Application Data
(60) Provisional application No. 61/583,977, filed on Jan. 6, 2012.
(60) Provisional application No. 61/673,197, filed on Jul. 18, 2012.
(60) Provisional application No. 61/673,715, filed on Jul. 19, 2012.

(51) Int. Cl. C12Q 1/68

(52) U.S. Cl. CPC C12Q 1/6879; C12Q 2600/156; C12Q 2600/124
USPC 435/6.11

(57) ABSTRACT

Disclosed are sperm cells and embryos comprising a labeled locked nucleic acid bound to a gender-specific repeat sequence. Methods for identifying and separating sperm cells or embryos containing a labeled locked nucleic acid from sperm cells or embryos not containing the labeled oligonucleotide produce gender-enriched sperm cell or embryo fractions. The separated fractions are useful in producing offspring of a predetermined sex.

### RELATED APPLICATIONS

This application is a National Phase entry of PCT Application No. PCT/US2013/020139 filed Jan. 3, 2013, which claims priority from U.S. Provisional Patent Application No. 61/583,977, filed Jan. 6, 2012, U.S. Provisional Patent Application No. 61/673,197, filed Jul. 18, 2012, and U.S. Provisional Patent Application No. 61/673,715, filed Jul. 19, 2012, the disclosures of which are hereby incorporated by referenced herein in their entirety.

### INTRODUCTION

The production of offspring of a predetermined sex, or in a predetermined sex ratio, is desirable in a number of industries, including animal husbandry. The gender-specific separation of sperm cells or embryos may facilitate the production of offspring having a predetermined sex. Separated sperm cells may be used in artificial insemination or in vitro fertilization to produce zygotes that develop into organisms of a predetermined sex. However, techniques to produce populations of sperm cells or embryos that are sufficiently gender enriched are lacking.

### SUMMARY

In one aspect, a method for separating a population of sperm cells or embryos by contacting the population with a labeled locked nucleic acid capable of binding a gender-specific tandem repeat sequence that occurs in a portion of the population is provided. The labeled sperm cells or embryos are then separated from the unlabeled sperm cells. In one aspect, the locked nucleic acid is an invader locked nucleic acid. In one aspect, the locked nucleic acid is labeled with a fluorescent tag, a heavy density tag, a magnetic tag, a nanoparticle, a toxin, a DNA, a siRNA, an enzyme, a specific ion, and combinations thereof.

In one aspect, a sperm cell or embryo having a gender-specific tandem repeat sequence and a labeled oligonucleotide moiety, such as a locked nucleic acid, bound to the gender-specific sequence is provided.

In another aspect, a population of sperm cells or embryos having a gender-specific tandem repeat sequence occurring on the X or the Y chromosome is provided. A portion of the population of sperm cells or embryos have a labeled oligonucleotide moiety, which is a locked nucleic acid, bound to the gender-specific sequence.

In one aspect, a population of sperm cells having a gender-specific tandem repeat sequence occurring on the X or the Y chromosome is provided. A portion of the population of sperm cells have a labeled oligonucleotide moiety, which is a locked nucleic acid, bound to the gender-specific sequence to provide a labeled fraction and an unlabeled faction. In one aspect, at least 30% of the cells comprise a locked nucleic acid bound to the gender-specific sequence. In one aspect, at least 70% of the cells comprise the locked nucleic acid bound to the gender-specific sequence. In one aspect, at least 90% of the cells comprise the locked nucleic acid bound to the gender-specific sequence.

In one aspect, at least 70% of the sperm cells of the labeled fraction comprise a Y chromosome and at least 70% of the sperm cells of the unlabeled fraction comprise an X chromosome. In another aspect, at least 70% of the sperm cells of the labeled fraction comprise an X chromosome and at least 70% of the sperm cells of the unlabeled fraction comprise a Y chromosome. In one aspect, at least 50% of the sperm cells of the labeled fraction or the unlabeled faction are viable after the labeled fraction and the unlabeled fraction are separated. In one aspect, the sperm cells are separated by physical separation by flow cytometry, centrifugation, magnetic force, or chemical separation using processes affecting metabolism, viability, motility, integrity or fertility.

In one aspect, the sex of an embryo is identified by contacting at least one cell of the embryo with a locked nucleic acid. The locked nucleic acid comprises a label and is capable of binding a gender-specific tandem repeat sequence present in the cells of either the female or the male embryo. Detecting the presence or absence of the label in the embryo facilitates identifying the sex of the embryo. In one aspect, at least one cell of the embryo is viable. In one aspect, each cell of the embryo is contacted with the locked nucleic acid. In one aspect, the embryo comprises the locked nucleic acid bound to the gender-specific tandem repeat sequence and wherein the embryo is viable. In one aspect, the label comprises CY3 and wherein the label is detected using fluorometric techniques.

In one aspect, the sperm cells or embryos are permeabilized prior to the population being contacted with the labeled locked nucleic acid. In one aspect, the sperm cells or embryos are permeabilized using electroporation, liposomes, osmotic pressure, or permeating peptides.

In one aspect, micro, nano or other particles are used to facilitate penetration of the locked nucleic acid into the sperm cells or embryos prior to the population being contacted with the labeled locked nucleic acid.

In one aspect, the gender-specific tandem repeat sequence comprises a telomeric sequence. In one aspect, the gender-specific tandem repeat sequence is from about 2,000 to about 10,000 nucleotides.

In one aspect, the population comprises mammalian sperm cells or mammalian embryos selected from bovine, porcine, canine, and equine.

In one aspect, a method for targeting sequence-specific DNA with locked nucleic acids, such as for use in site-specific modulation of gene expression, or induction of site-specific genomic DNA changes (including mutation, recombination or repair) in living cells.

### BRIEF DESCRIPTION OF THE DRAWINGS

FIG. 1 is a schematic representation showing the location of gender-specific tandem repeat sequences (GSTRSs) and target sequences on a chromosome.

FIG. 2 is a repeated non-expressed sequence of the bovine Y chromosome showing the location of tandem repeat sequences (GSTRSs).

FIG. 3 is a drawing showing the structure of a suitable pyrene-functionalized locked nucleic acid and the functioning of invader locked nucleic acids with double stranded nucleotides. Tₑₓₚ is the experimental temperature and Tₘ is the dissociation temperature.

FIG. 4 is a photograph showing male bovine somatic nuclei and Invader LNA.

FIG. 5 is a photograph showing invader LNA-Cy3 on a fixed male bovine embryo.

FIG. 6 is a photograph showing a live Bovine embryo labeled with INV-Cy3 probe and co-labeled with Hoechst 33342 to show the co-localization of INV-Cy3 in Hoechst labeled nuclei.

FIG. 7 is a photograph showing fixed boar sperm hybridized to an iLNA probe specific for a y-chromosome sequence.

### DETAILED DESCRIPTION

The invention relates to the identification of the sex of sperm cells and embryos and the generation of sperm cell fractions or embryo fractions that are enriched for the X or the

Y chromosome. In one embodiment, the invention provides methods for separating sperm cells that contain a labeled oligonucleotide moiety (a locked nucleic acid) bound to a gender-specific tandem repeat sequence or a complement of a gender-specific tandem repeat sequence. The oligonucleotide moieties suitably bind in sufficient numbers to a region of the chromosome to generate a detectable signal that can be used as a basis for distinguishing, and optionally separating cells that contain the gender-specific tandem repeat sequence from those that do not. The invention further provides a method for the separation of sperm cells or embryos carrying an X chromosome from sperm cells or embryos carrying a Y chromosome. The gender-enriched sperm cell fractions can be used to fertilize ova to produce offspring of a predetermined sex. The invention further provides a method for selection of embryos carrying an X chromosome or embryos carrying a Y chromosome. The embryos that have been contacted with the labeled locked nucleic acid are suitably viable, such that the destruction of one or more cells of the embryo is avoided.

In another aspect the invention relates to the ability to target sequence-specific DNA that can be used for site-specific modulation of gene expression, induction of specific genomic DNA changes (including mutation, recombination or repair) in living cells by the specific binding and activation of a locked nucleic acid.

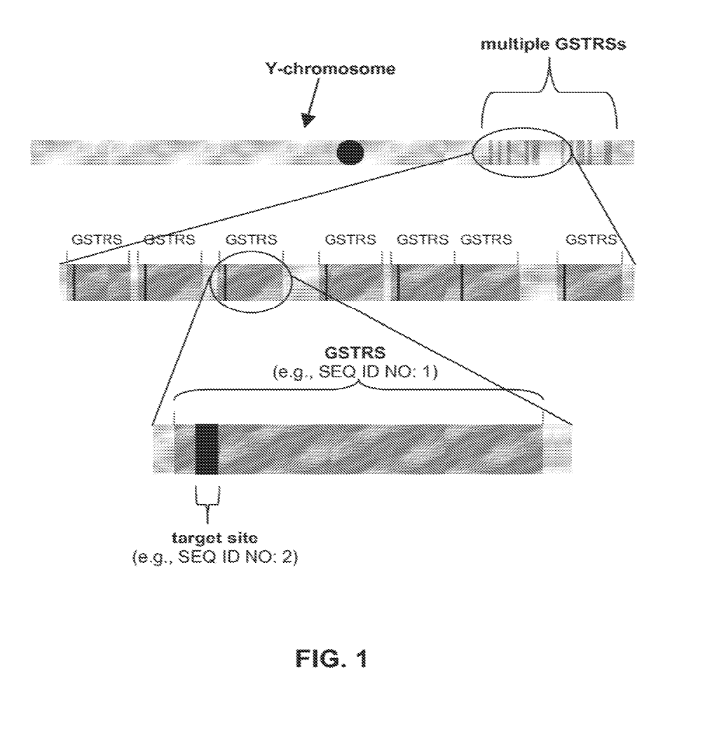

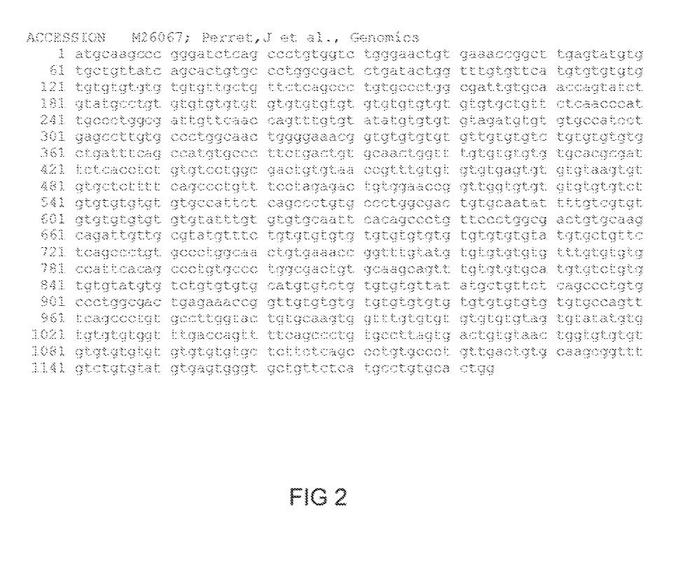

As used herein, a “gender-specific tandem repeat sequence,” or “GSTRS” is a non-autosomal chromosome sequence that is repeated on either the Y chromosome or the X chromosome, but not both. Multiple GSTRSs occur in a region of the X or Y chromosome, as shown schematically in FIG. 1. FIG. 2 shows a repeated non-expressed sequence of the bovine Y chromosome showing the location of tandem repeat sequences (GSTRSs). The GSTRS may occur anywhere on the X or Y chromosome. In some embodiments, the GSTRS targets of the invention occur at or near the termini of the chromosome. The gender-specific tandem repeat sequence may comprise at least about 10 nucleotides, at least about 50 nucleotides, at least about 100 nucleotides, at least about 500 nucleotides, at least about 1,000 nucleotides, at least about 2,000 nucleotides, at least about 3,000 nucleotides, or at least about 4,000 nucleotides, and less than about 10,000 nucleotides, less than about 9,000 nucleotides, less than about 8,000 nucleotides, less than about 7,000 nucleotides, less than about 6,000 nucleotides, or less than about 5,000 nucleotides. Suitably there are less than about 50,000 nucleotides, about 10,000 nucleotides, about 5,000 nucleotides, about 3,000 nucleotides, about 2,000 nucleotides, about 1,000 nucleotides, about 500 nucleotides, about 300 nucleotides, about 100 nucleotides, about 10 nucleotides, about 1 nucleotide, or zero nucleotides between each unit of the repeated GSTRS. The GSTRS does not have to be repeated as exactly the same sequence, and some variation in the repeated sequences is possible without affecting the scope of the invention. The units of repeated GSTRSs may share at least about 70%, at least about 80%, at least about 85%, at least about 90%, at least about 95%, at least about 97%, at least about 98%, or at least about 99% identity with each other. Percent identity may be determined using algorithms used in BLASTn or MEGABLAST programs, which may be used to obtain sequences homologous to a reference polynucleotide, as is known in the art. Algorithms used for sequence alignment are described by Tatiana A. Tatusova, Thomas L. Madden (1999), FEMS Microbiol Lett. 174:247-250. The GSTRS may be repeated at least about 50 times, at least about 100 times, at least about 200 times, at least about 300 times, at least about 400 times, at least about 500 times, at least about 750 times, or at least about 1000 times on a chromosome.

For each GSTRS, a locked nucleic acid may be selected to bind to the GSTRS or a complement of the GSTRS. As depicted schematically in FIG. 1, the locked nucleic acid may target a shorter target sequence within the GSTRS. As used herein, “target sequence” is a segment of DNA within the GSTRS, wherein the locked nucleic acid binds the target sequence or the complement of the target sequence. The target sequence may include at least about 4, at least about 6, at least about 8, at least about 10, at least about 12, at least about 14 nucleotides, at least about 16 nucleotides, or at least about 18 nucleotides. The target sequence may include less than about 100, less than about 90, less than about 80, less than about 70, less than about 50, less than about 40, less than about 30, less than about 20, or less than about 16 nucleotides. The locked nucleic acid may bind to at least about 4 nucleotides, at least about 5 nucleotides, at least about 6 nucleotides, at least about 9 nucleotides, at least about 12 nucleotides, at least about 15 nucleotides, at least about 20 nucleotides, at least about 25 nucleotides, at least about 30 nucleotides, or at least about 35 nucleotides of the GSTRS or the complement of the GSTRS. The locked nucleic acid may bind to less than about 100 nucleotides, less than about 50 nucleotides, less than about 45 nucleotides, less than about 40 nucleotides, or less than about 20 nucleotides of the GSTRS or the complement of the GSTRS.

Suitable GSTRSs may be selected by searching public databases for DNA sequences that are highly repetitive on only the X or the Y chromosome. Suitable target sequences within the GSTRS may be selected by scanning the GSTRS for consecutive purines or consecutive pyrimidines, for example, homopurine or homopyrimidine sequences.

Homopurine or homopyrimidine sequences facilitate binding of oligonucleotide moieties such as locked nucleic acids to the major groove of duplex DNA to form a triplex. The target sequence within the gender-specific tandem repeat sequence may include, but is not limited to, homopurine or homopyrimidine sequences, as in certain embodiments, locked nucleic acids are capable of binding DNA of any sequence, including mixed DNA sequences that include all different nucleotides, and not only homopurines or homopyridamines.

In some embodiments, the target sequence is itself a repeated unit within the GSTRS. The GSTRS may include at least about 1, at least about 2, at least about 3, at least about 4, at least about 5, at least about 7, at least about 10, at least about 15, at least about 50, at least about 100, or at least about 200 repeated units of target sequence. A GSTRS having a higher number of repeated units will facilitate binding of more oligonucleotide moieties to the GSTRSs. Suitably, at least about 5, at least about 10, at least about 100, at least about 200, at least about 300, at least about 400, at least about 500, at least about 1,000, at least about 5,000, at least about 25,000, or at least about 50,000 oligonucleotide moieties bind to the GSTRSs.

In some embodiments, more than one target sequence may be selected within the GSTRS, or a complement of the GSTRS. A GSTRS having a higher number of target sequences will facilitate binding of more oligonucleotide moieties to the GSTRSs. In other embodiments, more than one type of locked nucleic acid may be selected to bind the GSTRS or a complement of the GSTRS.

The oligonucleotide moiety that binds to the target sequence is a locked nucleic acid (LNA), and particularly suitable are invader locked nucleic acids (iLNA). Locked nucleic acids (LNAs) are modified RNA nucleotides modified with an extra bridge which “locks” the ribose in the 3′-endo (North) conformation. LNA nucleotides can be mixed with DNA or RNA residues. Such oligonucleotides moieties are typically synthesized chemically. The locked ribose conformation enhances base stacking and backbone pre-organization. This significantly increases the hybridization properties (melting temperature) of oligonucleotides.

Invader LNAs are DNA duplexes with “+1 interstrand zipper arrangements” of intercalator-functionalized 2′-amino-alpha-1-LNA monomers. Invader LNAs facilitate sequence-unrestricted targeting of double stranded DNA (dsDNA) at physiologically relevant conditions and are able to specifically recognize short mixed sequence dsDNA targets.

Current probe technologies, such as TFO (triplex forming oligonucleotides) and PNA (peptide nucleic acids), typically experience target sequence restrictions and/or require non physiological ionic strengths for efficient dsDNA recognition. Invader LNAs, which are duplex probes with energetic hotspots consisting of +1 interstrand zippers of N2-pyrenefunctionalized 2′-amino-alpha-L-LNA monomers, enable efficient targeting of isosequential dsDNA. iLNA nucleotides increase the strength, sensitivity and specificity of techniques based on oligonucleotides and facilitate sequence specific targeting of mixed sequence of dsDNA at physiological conditions.

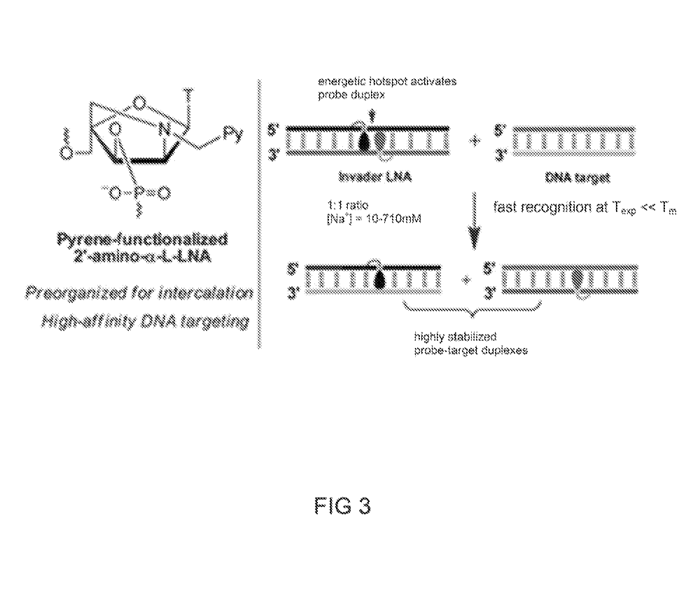

LNA and iLNA can be chemically synthesized. Suitable methods for synthesizing LNAs and iLNAs are described in PCT Publication No. WO2011/032034, which is herein incorporated by reference in its entirety. FIG. 3 illustrates the structure and functioning of a suitable invader LNA. The locked nucleic acids may show increased binding affinity toward double stranded DNA (via Hoogsteen base-pairing), single stranded DNA (via Watson-Crick base-pairing), and single stranded RNA targets (via Watson-Crick base-pairing). The locked nucleic acids improve discrimination of mismatched nucleic acid targets to minimize false positives and non-target specific effects in diagnostic and biological applications. The locked nucleic acids may also enhance stability against degradation by enzymes, such as nucleases.

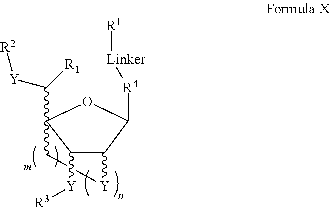

A C5 functionalized nucleotide suitable for use in a locked nucleic acid for use in the methods and compositions disclosed herein is shown in Formula X. With respect to Formula X, R¹ can be selected from hydrogen, hydroxyl, thiol, aliphatic, heteroaliphatic, aryl, heteroaryl, charged moieties, and metal complexes. R² can be selected from hydrogen, aliphatic, heteroaliphatic, aryl, heteroaryl, functional group protecting groups, a heteroatom-containing compound, such as a phosphorus-containing compound, a nitrogen-containing compound, an oxygen-containing compound, a sulfur-containing compound, and a selenium-containing compound. R³ can be selected from hydrogen, a heteroatom-containing compound, such as a phosphorus-containing compound, a nitrogen-containing compound, an oxygen-containing compound, a sulfur-containing compound, and a selenium-containing compound. R⁴ is a nucleobase selected from natural or non-natural nucleobases. The linker moiety can be selected from aliphatic, aryl, heteroaliphatic, and heteroaryl. Y can be selected from oxygen, sulfur, or NR⁵ where R⁵ is selected from hydrogen, aliphatic, aryl, heteroaliphatic, and heteroaryl; and m+n=2 to 4.

In certain embodiments, R¹ can be selected from ether, carbonyl, nitrile, disulfide, thioether, amine, amino acid, aminoglycoside, carbohydrate, fluorophores, nucleosides, nucleotides, oligonucleotides, peptides, intercalators, lipidoids, sterols, porphyrins, proteins, and vitamins. In particular embodiments, R¹ can be selected from amide, ester, carboxylic acid, aldehyde, ketone, spermine derivatives, guanidine groups, spin labels, electrochemical probes, fatty acids, glycerols, glycols, polyethylene glycol, redox active FRET labels, and ferrocene derivatives. Even more typically, R¹ can be selected from hydrogen, hydroxyl, thiol, primary amine, biotin, lauric acid, palmitic acid, stearic acid, fluorescein, rhodamine, cyanine, pyrene, perylene, coronene, adamantine, acridine, phenantroline, diphenylphosphorylazide, HIV Tat fragment, transportan, cholesterol, lithocolic-oleyl, myristoyl, docosanyl, lauroyl, stearoyl, palmitoyl, oleoyl, and linoleoyl, dihydrotestosterone, lithocholic acid, folic acid, and vitamin E.

In certain embodiments the monomer is of Formula Y

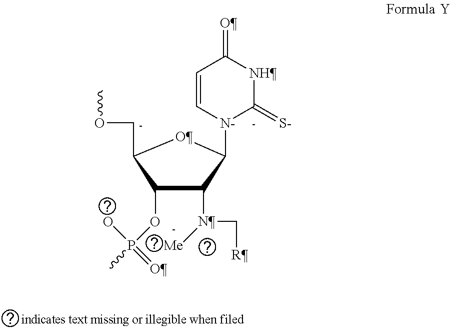

The locked nucleic acid that binds to the GSTRS may include a label that is detectable when bound to the gender-specific tandem repeat sequence. Suitable labels include, but are not limited to, dyes, fluorescent molecules such as CY3 or CYS, molecules of heavy density such as gold or iron, magnetic molecules, nanoparticles, picoparticles, or any combination thereof. The labeled locked nucleic acid binds in sufficient numbers to the GSTRSs to produce a detectable signal. The signal may be detectable by any suitable method including, but not limited to, centrifugation, fluorescence, luminescence, microscopy, magnetic force, densitometry, or combinations thereof. Methods of coupling labels to oligonucleotides are known in the art and can be adapted for coupling to the locked nucleic acids described herein.

The locked nucleic acid that binds to the GSTRS may include a reactive chain that is activated when bound to the gender-specific tandem repeat sequence and that can affect, for example, DNA integrity, cell metabolism, viability, motility, fertility, or a combination thereof. Suitable reactive groups include, but are not limited to, amine linkers, toxins, RNA sequences, DNA sequences, enzymes, nanoparticles, picoparticles, or any combination thereof. The activated locked nucleic acid binds in sufficient numbers to the GSTRSs to produce a chain reaction. The chain reaction may affect cell DNA integrity, cell viability, cell motility, metabolism, fertility, and may allow segregation of the targeted cell population from the non-bound cell population and from there allow separation, segregation, or discrimination of the cell population, thus affecting the sex ratio after fertilization. Methods of coupling labels to oligonucleotides are known in the art and can be adapted for coupling to the locked nucleic acids described herein.

In other embodiments, locked nucleic acids may be labeled with labels that are active for fluorescence resonance energy transfer (FRET) or for conditional release activation of specific reactive groups (CRA). Some locked nucleic acids may be labeled with a FRET or CRA donor, and others may be labeled with a FRET or CRA acceptor. Excitation of the donor label may excite the acceptor label, and cause the acceptor label to fluoresce or to release the activated group. FRET may thus be used to enhance or differentiate the signal of the labeled locked nucleic acids bound to GSTRSs in proximity on the chromosome and improve signal to noise ratio. CRA may thus be used to affect, inhibit, or modify the integrity, metabolism, motility, viability, or fertility of the targeted/activated cell. For example, two locked nucleic acids can be designed to bind to a target sequence so that the locked nucleic acids are located close to each other after binding to the target sequence, e.g., a first locked nucleic acid may be designed to bind base pairs 1 to 12 and a second locked nucleic acid may be designed to bind base pairs 13 to 24 of a target sequence 24 base pairs in length. When the two different locked nucleic acids are labeled with suitable dye molecules, for example a cyan fluorescent protein (CFP) as donor and yellow fluorescent protein (YFP) as acceptor, FRET may be used. The labeled cells may be excited with light of a suitable wavelength. For example, if excited with a wavelength of 440 nm, CFP will emit light at 480 nm wavelength which overlaps with the excitation wavelength of YFP, and will lead to a YPF signal emission peak at 535 nm when both locked nucleic acids are close together. After activation the process may also release active groups, toxics, RNA, DNA, enzymes, affecting, but not limited to, life, metabolism, motility and fertility of the targeted cell

In another embodiment, the label may suitably be a molecule, such as DNA or RNA, or atom attached to the locked nucleic acid that enhances activation or deactivates physiological process of the cell, and may be toxic and/or facilitates destruction, incapacitation or inactivation of the cell when bound to a GSTRS. For example, a cell toxin when attached to the GSTRS, may cause the cell to die, may facilitate impairment of the functioning of the cell, may disrupt the cell physiologically, or may impair cellular integrity, so that the cell becomes unviable or incapacitated. Mechanisms through which the label may affect the cell include, without limitation, an increase in intra-cellular pH, an accumulation of cell toxins, induction of selective phototoxicity, impairment of mitochondrial function, altered cell motility, inducement of acrosome reaction, cell death through a direct cellular action or the action of electromagnetic waves on the label and combinations thereof. The enriched sperm cell fractions may be thus be generated without needing to separate a viable population of labeled cells from a viable population of unlabeled cells. Such a label may be used in conjunction with, or independently from, one or more detectable labels bound to the same or other locked nucleic acids.

Suitably, the molecule or atom that facilitates destruction or incapacitation of the cell functions effectively when in proximity to other labels, which labels may be the same or different, and which may each be attached to separate locked nucleic acids , as would occur upon binding of the to the GSTRS.

Labels may also be used which regulate the capacitation, viability, motility, fertility or combination thereof of sperm cells containing a GSTRS. Accordingly, the timing at which a labeled sperm cell containing the GSTRS has the capacity to fertilize an egg may be controlled. For example, a sperm cell may be incapacitated in its ability to fertilize an oocyte, by inducing premature capacitation, by affecting cell motility or motility pattern, or by inducing apotosis or cell death. Fertilization of an egg can then be delayed by an appropriate amount of time, such that the labeled fraction of cells in the population is unable to fertilize the egg.

Suitable labels which may be used include, for example, noble metals such as silver, gold, platinum, palladium, rhodium, and iridium, and alloys and molecules thereof, as well as magnetic compounds. Suitable labels may also include siRNA, ions, proteins, peptides, and labels activated after release to affect cell integrity, viability, motility or fertility. Suitably these labels may be attached as picoparticles or nanoparticles. Cells labeled with such metals or compounds may subsequently be exposed to electromagnetic radiation, such as sono- or radiowaves, which may heat and/or excite the label resulting in the viability of the cell being impaired or reduced. Other suitable labels include calcium or calcium-containing compounds, calcium/ion pump activators, hydrogen ion/pH pump activators, organic compounds with alcohol groups, acids, and denaturing enzymes such as trypsin.

Labels may be attached to oligonucleotides using techniques known in the art for generally coupling molecules to oligonucleotides.

In a further embodiment, methods for distinguishing and separating sperm cells or embryos that contain a locked nucleic acid bound to a GSTRS. In some embodiments, the sperm cells or embryos are mammalian. Suitably, the sperm cells or embryos are mammalian, piscian or avian, or from vertebrates. The sperm cells may be of porcine, equine, bovine, ovine, caprine, feline, canine, or human origin. In other embodiments, the sperm cells or embryos are piscian or avian. As used herein, a “population” of sperm cells or embryos means at least two sperm cells or at least two embryos. However, the technology can also be used to identify and specifically label an individual embryo (such as for sex determination) or sperm cell (such as to perform ICSI).

In a first step of the method to identify gender or to generate gender-enriched sperm cell or embryo fractions, after buffer washing and equilibration, cells are contacted with the labeled oligonucleotide moiety. In some embodiments, the cell or cells are permeabilized to facilitate entry of the oligonucleotide into the cells and access to the GSTRS. The cells may be permeabilized by any suitable technique, including but not limited to, osmotic pressure, electroporation, liposomes, permeating peptides, a modified (for example increased or decreased) temperature or combinations thereof. In other embodiments, the labeled locked nucleic acid is passively or actively transported into the cell. The locked nucleic acid may further include a transport moiety, such as a transport peptide, micro or nanoparticles, which facilitates or mediates active uptake of the locked nucleic acid into the cell. Suitable transport peptides are commercially available from AnaSpec (San Jose, CA, U.S.A.) and include Arg9, TAT, and Cys-TAT. Transport peptides compatible with the ergothionine transporter may also be used.

Once the locked nucleic acids are bound to the repeated DNA sequence, the sperm cells may be identified and/or separated. The clustering of the labeled locked nucleic acid in the region of the GSTRS produces a signal (physical, optical or chemical) that may be detectable and enables cells that contain the GSTRS to be distinguished from cells that do not contain the GSTRS or not but induces a physical, chemical or other reactions that enables cells that contain the bound GSTRS to i.e. but not exclusive, be specifically affected in their integrity, viability, motility, metabolism, fertility or any combination thereof. Once labeled, the cells may be either detected or separated, or both detected and separated. Suitable methods for separating cells include, but are not limited to, micromanipulation, centrifugation, magnetic force, flow cytometry, densitometry, or chemical agents that induce changes in metabolism, viability, motility, integrity, fertility or any combination thereof. Suitably, at least about 25%, at least about 30%, at least about 40%, at least about 50%, at least about 60%, at least about 70%, at least about 80%, at least about 90%, or at least about 95% of the cells in the separated population of cells comprise a labeled locked nucleic acid bound to the GSTRS. The population of cells may be separated into a labeled fraction that contains the GSTRS, and an unlabeled fraction that does not contain the GSTRS.

In one embodiment, the labeled fraction includes sperm cells containing an X chromosome labeled with the oligonucleotide, and the unlabeled fraction includes sperm cells containing Y chromosome not labeled with oligonucleotide. In another embodiment, the labeled fraction includes sperm cells containing a Y chromosome labeled with the oligonucleotide, and the unlabeled fraction includes sperm cells containing an X chromosome not labeled with oligonucleotide. Suitably, a fraction may contain sperm cells wherein at least about 60%, at least about 65%, at least about 70%, at least about 75%, at least about 80%, at least about 85%, at least about 90%, at least about 95%, or at least about 99% of the sperm cells comprise an X chromosome. Alternatively, a fraction may contain sperm cells wherein at least about 60%, at least about 65%, at least about 70%, at least about 75%, at least about 80%, at least about 85%, at least about 90%, at least about 95%, or at least about 99% of the sperm cells comprise a Y chromosome.

The separated fractions suitably contain viable sperm cells. As used herein, “viable” refers to a sperm cell that is able to fertilize an egg to produce an embryo. Suitably, a separated sperm fraction contains sperm wherein at least about 20%, at least about 30%, at least about 40%, at least about 50%, at least about 60%, at least about 70%, at least about 80%, at least about 90%, at least about 95%, or at least about 99% of the sperm cells are viable.

A gender-enriched sperm cell fraction may be used to fertilize an egg in vitro or in vivo. Fertilization of an egg may be accomplished, for example, via artificial insemination, including, but not limited to, intra-vaginal, intra-cervical, intra-uterine or surgical insemination, or by intracytoplasmic sperm injection (ICSI). A labeled fraction or unlabeled fraction may be used for in vivo or in vitro fertilization. The fertilized egg may be allowed to develop to produce an embryo of predetermined sex.

In a further embodiment, the invention provides methods for determining the sex of embryos. Labeled locked nucleic acids designed to bind to a GSTRS as described above may be incubated with embryos, enter the embryos, and bind the GSTRS. As with sperm cells, the embryos may be permeabilized to facilitate entry of the labeled locked nucleic acid into the embryos. One or more cells from the embryo may also be removed or biopsied and permeabilized to facilitate entry of the labeled oligonucleotide. The sex of the biopsied cells may then be correlated with the embryo from which the cells were removed. The labeled oligonucleotide moiety can also be used to mark and identify live embryo without affecting live embryo development and fertility. Once the locked nucleic acid is bound to the GSTRS, the embryos may be viewed under a dissecting microscope or fluorescent microscope to distinguish embryos that contain the GSTRS from those that do not contain the GSTRS. As with sperm cells as described above, the population of embryos may be separated into a labeled fraction that contains the GSTRS, and an unlabeled fraction that does not contain the GSTRS.

The following examples are provided to assist in a further understanding of the invention. The particular materials and conditions employed are intended to be further illustrative of the invention and are not limiting upon the reasonable scope of the appended claims.

### EXAMPLES

### Example 1

### Porcine GSTRSs, Target Sequences, and Corresponding Oligonucleotide Moieties

Two complementary iLNA (duplex) designated “Sequence A” and “reversed Sequence A” each bind the target and reverse target sequence shown in SEQ ID NO: 2 of double-stranded DNA. SEQ ID NO: 2 is a 14-nucleotide sequence at nucleotide positions 3231 to 3244 of the GSTRS depicted in SEQ ID NO: 1. SEQ ID NO: 1 occurs on the porcine Y chromosome and has sequence Accession number X12696 (McGraw et al. (1988) Nucleic Acids Research, volume 16, page 10389). Sequence A and Reversed Sequence A were each synthesized with a CY3 fluorescent molecule attached to the 5′-end by an ester bond. Sequences A and reversed A were custom-ordered Prof. P,. Hrdlicka Biorganic chemistry of the University of Idaho. iLNAs were received as a lyophilized powder, and they were resuspended in ultrapure water and stored in aliquots at 20° C. and at 80° C.

A somatic tandem repeated DNA sequences was identified on porcine chromosome 1 with sequence Accession number X51555 (SEQ ID NO: 3). It is a 313 base pair DNA sequence that is repeated approximately 3000 to 6000 times. Two iLNAs designated “Sequence B ” and “reversed Sequence B” were each designed to bind to the target sequence shown in SEQ ID NO: 4. SEQ ID NO: 4 is a 14-nucleotide sequence at nucleotide positions 120 to 133 of the tandem repeat DNA sequence shown in SEQ ID NO: 3. Both Sequence B and Reversed Sequence B were each synthesized with a CY3 fluorescent molecule linked to the 5′-end by an ester bond. This somatic DNA sequence (SEQ ID NO: 3) was used as a negative control for experiments.

Examples of DNA sequences of porcine GSTRSs, target sequences, and corresponding oligonucleotides moieties are shown in Table 1 below.

TABLE 1

| Name | Description | DNA Sequence, 5′ → 3′ |
|---|---|---|
| SEQ ID | GSTRS on porcine | See SEQ ID NO: 1. |
| NO: 1 | Y chromosome. | |
| SEQ ID | Target sequence | 5′-CTCCTAAGTATGAC-3′ |
| NO: 2 | near the 5′-end | |
| | of the GSTRS | |
| | shown in SEQ ID | |
| | NO: 1. | |
| SEQ ID | Tandem repeat | See SEQ ID NO: 3. |
| NO: 3 | sequence on | |
| | porcine | |
| | chromosome 1. | |
| SEQ ID | Target sequence | 5′-TCCGCCTCCTCCCT-3′ |
| NO: 4 | in the tandem | |
| | repeat sequence | |
| | shown in SEQ ID | |
| | NO: 3. | |
| Sequence | iLNA; binds to | 5′-GAGGATTCATACTG-3′ |
| A | target direct | |
| | sequence shown | |
| | in SEQ ID NO: 2. | |
| Reverse | iLNA; | 5′-CTCCTAAGTATGAC-3′ |
| SequenceA | complementary | |
| | sequence to | |
| | Sequence A | |
| | binds to | |
| | target reverse | |
| | sequence | |
| | shown in | |
| | SEQ ID NO: 2. | |
| Sequence | iLNA; binds to | 5′-AGGCGGAGGAGGGA-3′ |
| B | target direct | |
| | sequence shown | |
| | in SEQ ID NO: 3. | |
| Reverse | iLNA; | 5′-TCCGCCTCCTCCCT-3′ |
| Sequence | complementary | |
| B | sequence to | |
| | Sequence | |
| | B binds | |
| | to target | |
| | reverse | |
| | sequence | |
| | shown in | |
| | SEQ ID NO: 3. | |

### Example 2

### Bovine GSTRSs, Target Sequences, and Corresponding Oligonucleotide Moieties

A 1399 base pair GSTRS was identified on the bovine X chromosome at locus V00125 (SEQ ID NO: 5) with sequence Accession number V00125. Two complementary iLNA (duplex) designated “Sequence C” and “reversed Sequence C” each bind the target and reverse target sequence shown in SEQ ID NO: 5 of double-stranded DNA. SEQ ID NO: 5 is a 20-nucleotide sequence at nucleotide positions 561 to 581 of the GSTRS shown in SEQ ID NO: 5. Sequence C and Reversed Sequence C were each synthesized with a CY3 fluorescent molecule attached to the 5′-end by an ester bond. Sequences C and reversed C were custom-ordered Prof. P,. Hrdlicka Biorganic chemistry of the University of Idaho. iLNAs were received as a lyophilized powder, and they were resuspended in ultrapure water and stored in aliquots at 20° C. and at 80° C.

TABLE 2

| | | |
|---|---|---|
| SEQ ID | GSTRS on bovine X | See SEQ ID NO: 5. |
| NO: 5 | chromosome. | |
| SEQ ID | Target sequence in | 5′-CACTATTATCGCCA |
| NO: 6 | the GSTRS shown in | TC-3′ |
| | SEQ ID NO: 5. | |
| Sequence | iLNA; binds to target | 5′-GTGATAATAGCGGT |
| C | sequence shown in | AG-3′ |
| | SEQ ID NO: 6. | |
| Sequence | iLNA; Complementary | 5′-CACTATTATCGCCA |
| Reversed | sequence to sequence | TC-3′ |
| C | C binds to reversed | |
| | target sequence shown | |
| | in SEQ ID NO: 6. | |

### Example 3

### Method of Labeling Fixed Porcine Sperm and Somatic Cells with CY3-iLNA Conjugate

Freshly-ejaculated boar semen or thawed boar semen (about 100 million sperm cells) was added to 10 mL of phosphate-buffered saline (PBS). The suspension was centrifuged for 5 minutes at 800×g. The pellet was resuspended in 1 mL of 3 M NaOH. The suspension was incubated at room temperature for 5 minutes and centrifuged for 5 minutes at 800'g. The pellet was resuspended in 2 mL of PBS and centrifuged for 5 minutes at 800'g. The pellet was resuspended in PBS or phosphate buffer (PB) to obtain a final concentration of 10 million sperm cells per mL of PBS.

A suspension of male somatic nuclei was prepared using standard methods as follows:
a. a suspension of male bovine somatic nuclei was prepared using standard methods of: hypotonic treatment of cells using KCl, centrifuging the cells and resuspending them in 3:1 methanol:acetic acid then storing them (−20 C) in this solution until ready for use.
b. 0.5 uL of the fixed nuclei were placed onto a plastic microscope slide
c. the nuclei were dried down and then the slide was heated to 60° C. for 2 min
d. the labeling buffer was prepared as follows:
   1. 500 uL of 10 mM Tris HCl+1 mM EDTA (i.e. standard TE buffer, pH 7.2) were added to a 1.5 mL microcentrifuge tube
   2. 0.5 uL of Invader LNA sequence A and reversed sequence A (from a stock solution of 50 uM conc in dH2O) were added
   3. the mixture was vortexed for 2-3 sec to mix, The solution was kept at room temp until needed.
e. 300 uL of the labeling buffer was added on top of the fixed nuclei
f. the slide was placed in a humidified environment at 37° C. for 3 hr
g. After incubation, the label buffer was washed with TE buffer at 37 C for 5 min
h. After washing, the slide was dried and then 3.0 uL of mounting medium containing DAPI (i.e. SlowFade with DAPI, Invitrogen) was added and the sample covered with a coverslip
i. The slide was placed on microscope stage using a microscope that has fluorescence capabilities
j. The samples were observed at 10× using DAPI filter to locate nuclei and then after switching to 40× and using the Cy3 filter to excite the Cy3 dye conjugated to the Invader LNA.

After pre-treatment of the sperm cells, CY3 labeled-LNA duplex as prepared in Example 1 (designated Sequence A and reversed A) was incubated with the sperm cells at a final iLNA concentration of 100 ng/mL for 2 hours at 38° C. Sperm cells were centrifuged for 5 minutes at 800×g, the pellet was resuspended in PBST (PBS with 0.05% Tween 20), and the suspension was incubated for 20 minutes at 38° C. The sperm cells were centrifuged for 5 minutes at 800×g, and the pellet was resuspended in PBS or PB. CY3 labeled (Sequence A/reverse A)-duplex iLNA-treated sperm cells (4 μL) were viewed under a Zeiss AxioSkop fluorescence microscope. DAPI stain was optionally added to the sample just before observation with the microscope. Selective binding of the CY3 labeled-iLNA to the Y chromosome of fixed boar semen was observed. Fixed boar sperm cells pretreated with NaOH and RNase A and incubated with Y-chromosome specific CY3-iLNA stained Y chromosomes red. Somatic porcine chromosomes treated with Y-chromosome specific CY3-iLNA were stained red. Somatic porcine chromosomes stained with DAPI that binds DNA and RNA and were stained blue. A merged image of somatic porcine chromosomes stained with DAPI and CY3-iLNA was generated. Y-chromosomes appeared to be stained pink, indicating selective binding of CY3-iLNA probe to the Y-chromosomes.

We found the signals present in 161 of 302 (53.3%) sperm to consist of a single, centrally-located, round fluorescent label in the sperm head. The signal was observed as a nice dot in the nuclei of all the male somatic cells

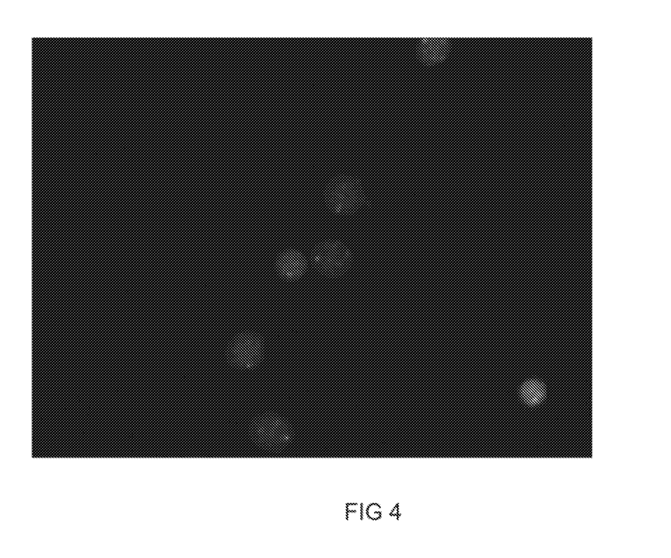

As a control, freshly-ejaculated boar semen was prepared and permeabilized as described above. A CY3-iLNA conjugate with base sequence (CCCTAA)₃, available from the Department of Chemistry of the University of Idaho that binds to the telomeres of all mammalian chromosomes was incubated with the resuspended sperm cells in PBS at a final iLNA concentration of 00 ng/μL for 2 hours at room temperature. CY3-iLNA (CCCTAA)₃-treated sperm cells (4 μL) were viewed under a Zeiss AxioSkop fluorescence microscope. Selective binding of CY3-iLNA (CCCTAA)₃ to all porcine chromosome telomeres of fixed boar semen was observed. Chromosomes stained with 4′,6-diamidino-2-phenylindole (DAPI) that non-specifically binds DNA and RNA appeared blue. In contrast, CY3-iLNA (CCCTAA)₃ stained chromosomes pink. FIG. 4 shows labeled male bovine somatic nuclei similarly labeled with invader LNA.

### Example 4

### Method of Labeling Live Bovine Sperm Cells with CY3-iLNA Conjugate

Freshly-ejaculated bull semen or thawed bull semen (about 100 million sperm cells) was added to 10 mL of phosphate-buffered saline (PBS). The suspension was centrifuged for 5 minutes at 800×g. The pellet was resuspended in PBS or phosphate buffer (PB) to obtain a final concentration of 10 million sperm cells per mL of PBS.

After pre-treatment of the sperm cells, CY3 labeled-LNA duplex (designated Sequence C and reversed C) as prepared in Example 1 was incubated with the sperm cells at a final iLNA concentration of 100 ng/mL for 2 hours at 38° C. Sperm cells were centrifuged for 5 minutes at 800×g, the pellet was resuspended in PBST (PBS with 0.05% Tween 20), and the suspension was incubated for 20 minutes at 38° C. CY3 labeled (Sequence C/reverse C)-duplex iLNA-treated sperm cells (4 μL) were viewed under a Zeiss AxioSkop fluorescence microscope. DAPI stain was optionally added to the sample just before observation with the microscope. Selective binding of the CY3 labeled-iLNA to the Y chromosome of bull semen was observed as red punctuated dots into the nucleus. In parallel killed bull sperm cells pretreated with NaOH and RNase A and incubated with Y-chromosome specific CY3-iLNA stained Y chromosomes similarly in red. Somatic porcine chromosomes treated with Y-chromosome specific CY3-iLNA also were stained red. Somatic porcine chromosomes stained with DAPI that binds DNA and RNA and were stained blue.

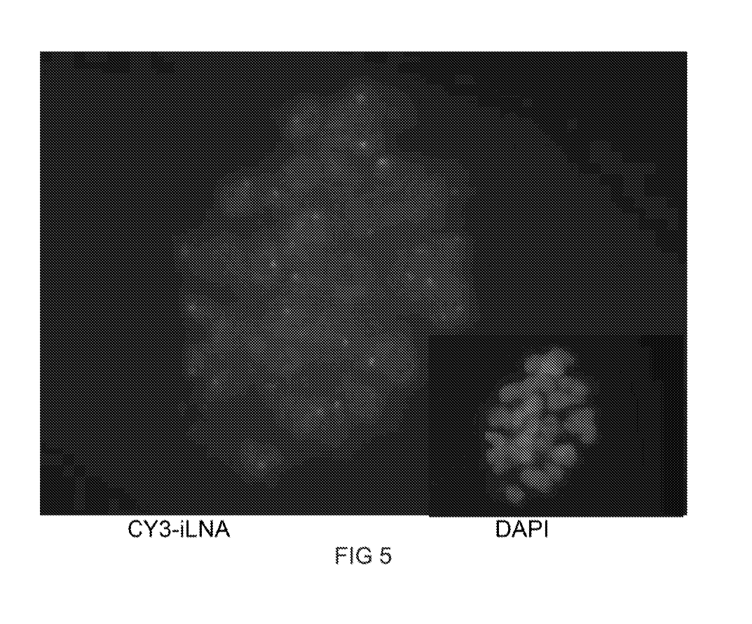

FIG. 5 shows Invader LNA-Cy3 on a fixed male bovine embryo.

### Example 5

### Sex Determination of Live Bovine Embryos

Fresh cultured blastocyst-stage bovine embryos (day 7 ideally) were washed with phosphate buffered saline (PBS) and were transferred into a 40 uL well of a microplate (ibidi) that was preloaded with 1× PBS pH 7.2

0.5 uL (100 ng) of iLNA sequence C and reversed C (from a stock concentration of 50 uM in dH2O) were added. The microplate containing the embryo was placed in a 37° C. incubator that was humidified and were incubated for 2.5 hr After incubation the embryos were transferred to another 40uL well containing 1× PBS without iLNAs The embryos were washed for 5 min at room temp, and then the microplate was placed onto a microscope stage using a microscope that is fitted with fluorescence capacity (Zeiss AxioSkop fluorescence microscope). The embryos are observed at 10× to locate, then viewed at 20× or 40× using fluorescence.

The iLNA duplex probe C and reversed C targets the unique Y-chromosome specific sequence SEQ ID NO. 5. Y-chromosomes were detected as a bright fluorescent red spot within the blastomer nuclei. The absence of signal indicated female embryonic DNA. The accuracy of the sexing procedure was demonstrated by parallel gender determination of the same embryo using an established PCR method designed for the bovine SRY male specific gene locus. Based on 18 in vitro produced bovine embryos generating a result for both assays, there was a 100% match (18/18) of gender assignment.

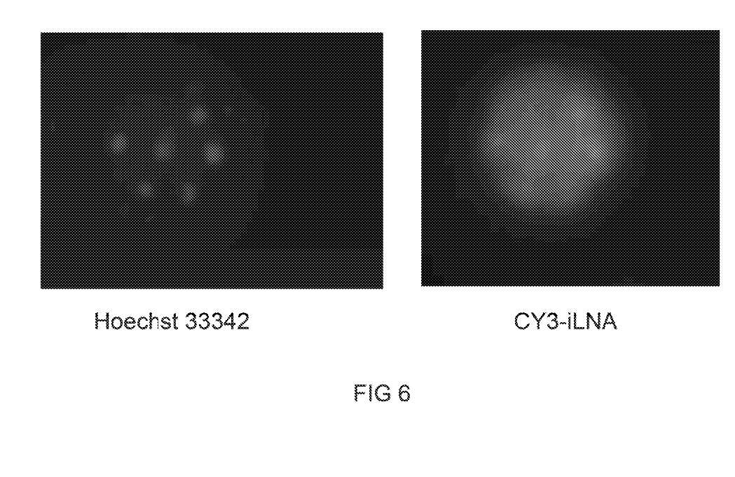

FIG. 6 shows live Bovine embryo labeled with INV-Cy3 probe and co-labeled with Hoechst 33342 to show the co-localization of INV-Cy3 in Hoechst labeled nuclei.

### Example 6

### In vitro Fertilization of Porcine Eggs with X or Y Chromosome-Enriched Boar Semen

Viable boar sperm cell fractions labeled with CY3-iLNA or unlabeled were used to fertilize porcine eggs. About 1.5 to 2 hours before preparing the semen, one plate or dish containing 5 to 10 mL of TALP media and one plate or dish containing 5 to 10 mL of FERT media (TALP+caffeine) were prepared and placed in an incubator 38.5° C. for at least 1.5 hours to equilibrate. Additionally, approximately 30 mL of semen saline (0.9% saline +BSA) was placed in a hood to warm to room temperature. Sperm vision counting chambers were warmed.

To prepare the semen, 2 to 3 mL of the X or Y chromosome-enriched sperm cell fraction was brought up to 10 mL with semen saline (0.9% saline +BSA). The suspension was centrifuged at 800× g for 3 minutes. The semen saline was pulled down to the sperm pellet, the volume brought up to 10 mL with fresh semen saline, the pellet resuspended in fresh saline, and the suspension centrifuged. The washing procedure may be repeated for a total of three times. The final sperm pellet was resuspended in 3 mL of TALP, mixed gently, and a small sample was removed for subsequent sperm motility and concentration determination.

To prepare frozen-thawed X or Y chromosome-enriched sperm cell fraction, a frozen straw of semen (0.5 cc) was placed in a 50° C. water bath for 10 seconds. The thawed sperm was then layered over a density gradient and centrifuged at 350× g for 10 minutes. The pellet was washed once in 2 mL of CellGuard (Minitube, Verona, Wis., U.S.A.) and centrifuged at 200× g for 10 minutes. The pellet was diluted and mixed gently in 1 mL of TALP media, and a small sample was removed for subsequent sperm motility determination. Sperm motility and concentration was determined using Sperm Vision (Minitube of America, Verona, Wis., U.S.A).

To fertilize oocytes, 10 μL of sperm in FERT media (at a concentration of 2.5×10⁵ sperm/mL) was added to a 500 μL well containing 50 oocytes. In vitro fertilization of porcine oocytes is also described in Rath et al. (J. Anim. Sci. 77:3346-3352 and Long, et al. (1999) Theriogenology 51:1375-1390), each of which is incorporated herein by reference in its entirety.

### Example 7

### Generation of a CY3-Labeled iLNA Conjugate and Use to Identify Male and Female Sperm

Synthetic DNA mimics conjugated to a fluorescent dye were used for in situ detection of Y chromosomes in metaphase preparations of bovine somatic cells and spermatozoa. Using male bovine somatic cells and the Y-chromosome as a template, a synthesis a CY3-conjugated iLNA was designed and custom synthesized.

A iLNA designated “Sequence C and Reversed C” was designed to bind to the target sequence shown in SEQ ID NO:6. SEQ ID NO:6 is a 20-nucleotide sequence at nucleotide positions 561 to 581 of the GSTRS shown in SEQ ID NO: 5. SEQ ID NO. 5 is a bovine Y chromosome sequence thought to be repeated 60,000 times (Perret, J. et al., 1990. A polymorphic satellite sequence maps to the pericentric region of the bovine Y chromosome; Genomics Vol 6 (3) pp 482-490). The iLNA probe designated “Sequence C and reversed C” was custom synthesized with a CY3 fluorescent molecule linked to the 5′-end by an ester bond: CY3-CAC TAT TAT CGC CAT C

Flow cytometry generated sexed bull sperm were evaluated with the iLNA probe (Sequence C) for accuracy of scoring. By testing different labeling conditions, it was found that brief incubation of metaphase chromosomes with the iLNA produced a localized signal on the Y-chromosome. The Y sorted sperm population showed labeling with the PNA probe in 104 signals on sperm heads out of 118 counted. The X sorted population showed labeling with the iLNA y specific probe in 8 signals on sperm heads out of 119 counted. In other tests, no signals were present when chromosomes of bovine female somatic cells were incubated with the iLNA probe.

The iLNA signals present in about 50% of sperm were found to consist of a single, centrally-located, round fluorescent dot in the sperm head. Unsorted bull sperm provided 23 signals out of 43 sperm heads (53.4%). The iLNA probe was also found to produce signal in male bovine somatic cell lines and in embryos with a similar ratio.

FIG. 7 shows boar sperm hybridized to iLNA probe specific for a y-chromosome sequence. The y-chromosome resides in the middle of the sperm head.

### Example 8

### Separation of Fluorescently Labeled Viable Sperm Cells Via Flow Cytometry

Semen will be resuspended in semen extender (AndroHep CellGuard for boar sperm, commercially available from Minitube of America, Verona, Wis., U.S.A.) to give approximately 1×10⁷ cells per mL. 1 ng of CY3-iLNA conjugate of Example 1 (Sequence A) will be added to 0.6 mL of sperm cell suspension. The suspension will be incubated at 38 C for 2 hours. Uptake of iLNA into the sperm will be verified by fluorescence microscopy.

The labeled sperm cells will be separated from the unlabeled sperm cells under flow with the following conditions: Boar sperm cells will be separated using a FACSVantage SE with DiVa option flow cytometer (BD Biosciences, San Jose, Calif., U.S.A.) with 100 mW of 488 nm light from a Coherent INNOVO 90C Argon ion laser. A 100 μm nozzle tip will be used at a sheath pressure of 12 psi. The sheath fluid used will be sterile Dulbecco's Phosphate Buffered Saline (DPBS, without Ca²⁺ or Mg²⁺, Sigma-Aldrich, St. Louis, Mo., U.S.A.). Detectors used will include FSC-A for forward scatter, SSC-A for side scatter, FL1-A with a 530/30 nm bandpass filter to detect any auto-fluorescent material, FSC-W for doublet-discrimination, and FL2-A CY3 detector with a 585/42 nm bandpass filter to detect the PNA with CY3 fluorescent label. A flow cytometry histogram illustrating the separation of labeled and unlabeled boar sperm cells will demonstrate selective binding of CY3-PNA (Sequence A) to the Y chromosome and separation of sperm with X chromosome from sperm with Y chromosome. At least 85% of the cells in the labeled fraction are expected to contain the Y chromosome. At least 85% of the cells of the unlabeled fraction are expected to contain the X chromosome. This will be validated using PCR of individual sperm cells tested for the presence of the SRY gene.

### Example 9

### Additional Probes for Binding to Bovine and Porcine Target Sequences

Tables 3-6 show probes suitable for binding to either bovine or porcine target sequences shown in FIG. 2 (bovine) or SEQ ID NO: 1, which occurs on the porcine Y chromosome and has sequence Accession number X12696 (McGraw et al. (1988) Nucleic Acids Research, volume 16, page 10389. Underlined nucleotides show the position of the functionalized nucleotides. Cy3 indicates the probe has been labeled with Cy3. The numbers 9 and 4 and N in the sequences shown in Table 6 indicate building blocks that destabilize the probe. Tables 3, 4 and 5 also provide the Tm (temperature at which 50% of the probes dissociate from their target or binding dissociation temperature) of the different probes for the targeted sequences as well the TA (affinity differential) of the probe versus the DNA duplex target. A positive TA indicates higher affinity of the probe for single stranded DNA. The higher the temperature differential, the stronger the binding to single stranded DNA.

TABLE 3
Bovine Probes

| | Upper | Lower | | | |
|---|---|---|---|---|---|
| | probe strand | probe strand | | DNA | |
| | vs DNA | vs DNA | Probe | Duplex target | TA |
| Duplex | Tₘ[ΔTₘ](° C.) | Tₘ[ΔTₘ](° C.) | Tₘ[ΔTₘ](° C.) | Tₘ (° C.) | (° C.) |
| 5′-AGC CCT GTG CCC TG | 69.5 [+9.0] | 74.5 [+14.0] | 58.0 [−2.5] | 60.5 | +25.5 |
| 3′-TCG GGA CAC GGG AC | | | | | |
| 5′-CCT GTG CCC TG | 59.5 [+9.0] | 65.5 [+15.0] | 48.0 [−2.5] | 50.5 | +26.5 |
| 3′-GGA CAC GGG AC | | | | | |
| 5′-CCT GTG CCC TG | 59.0 [+8.5] | 64.0 [+13.5] | 47.0 [−3.5] | 50.5 | +25.5 |
| 3′-GGA CAC GGG AC | | | | | |
| 5′-AGC CCT GTG CCC TG | 69.5 [+9.0] | 75.5 [+15.0] | 61.5 [+1.0] | 60.5 | +23.0 |
| 3′-TCG GGA CAC GGG AC | | | | | |
| 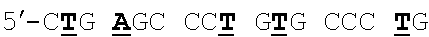 | 74.0 [+8.0] | 78.0 [+12.0] | 57.0 [−9.0] | 66.0 | +29.0 |
| 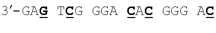 | | | | | |
| 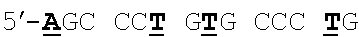 | 70.0 [+9.5] | 80.0 [+19.5] | 60.0 [−0.5] | 60.5 | +29.5 |
| 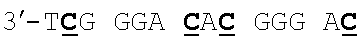 | | | | | |

TABLE 4
Additional Bovine Probes

| | Upper | Lower | | | |
|---|---|---|---|---|---|
| | probe strand | probe strand | | DNA | |
| | vs DNA | vs DNA | Probe | Duplex target | TA |
| Duplex | Tₘ[ΔTₘ](° C.) | Tₘ[ΔTₘ](° C.) | Tₘ[ΔTₘ](° C.) | Tₘ (° C.) | (° C.) |
| 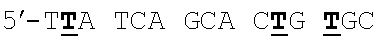 | 66.0 [+12.0] | 65.0 [+11.0] | 52.0 [−2.0] | 54.0 | +25.0 |
| 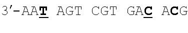 | | | | | |
| 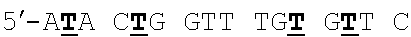 | 62.0 [+10.0] | 69.0 [+17.0] | 39.0 [−13.0] | 52.0 | +40.0 |
| 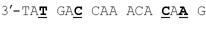 | | | | | |
| 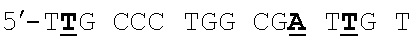 | 66.0 [+5.0] | 69.0 [+8.0] | 54.0 [−7.0] | 61.0 | +20.0 |
| 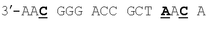 | | | | | |
| 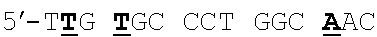 | 64.0 [+5.0] | 62.0 [+3.0] | very broad | 59.0 | N/A |
| 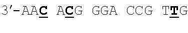 | | | | | |
| 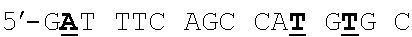 | 63.0 [+7.0] | 69.0 [+13.0] | 45.0 [−11.0] | 56.0 | +31.0 |
| 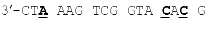 | | | | | |
| 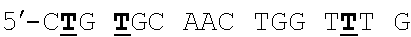 | 65.0 [+7.0] | 68.0 [+10.0] | 62.0 [+4.0] | 58.0 | +13.0 |
| 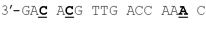 | | | | | |
| 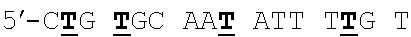 | 63.0 [+12.0] | 71.0 [+20.0] | 51.0 [±0] | 51.0 | +32.0 |
| 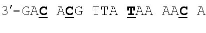 | | | | | |
| 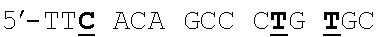 | 71.0 [+11.0] | 74.0 [+14.0] | 53.0 [−7.0] | 60.0 | +32.0 |
| 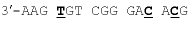 | | | | | |
| 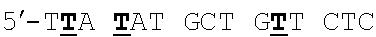 | 58.0 [+12.0] | 63.0 [+17.0] | 54.0 [+8.0] | 46.0 | +21.0 |
| 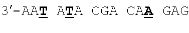 | | | | | |
| 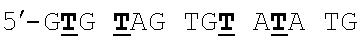 | 61.0 [+18.0] | 67.0 [+24.0] | 46.0 [+3.0] | 43.0 | +39.0 |
| 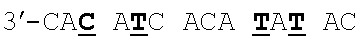 | | | | | |

TABLE 5
Porcine Probes

| | Upper | Lower | | | |
|---|---|---|---|---|---|
| | probe | probe | | | |
| | strand | strand | | DNA | |
| | vs DNA | vs DNA | Probe | duplex | |
| | Tₘ [ΔTₘ] | Tₘ[ΔTₘ] | Tₘ[ΔTₘ] | target | TA |
| Duplex | (° C.) | (° C.) | (° C.) | Tₘ(° C.) | (° C.) |
| 5′-GAC TAT TAG ACA CGA | 63.0 | 64.0 | 53.0 | 48.0 | +26.0 |
| 3′-CTG ATA ATC TGT GCT | [+15.0] | [+16.0] | [+5.0] | | |
| 5′-TCT ATA CTG TGT ATT C | 58.0 | 60.0 | ND | 43.0 | ND |
| 3′-AGA TAT GAC ACA TAA G | [+15.0] | [+17.0] | | | |
| 5′-CAC TAT TAT CGC CAT C | 66.0 | 67.0 | 58.0 | 53.0 | +22.0 |
| 3′-GTG ATA ATA GCG GTA G | [+13.0] | [+14.0] | [+5.0] | | |
| 5′-CCA TAG CCT AAG C | 66.0 | 62.0 | 53.0 | 46.0 | +29.0 |
| 3′-GGT ATC GGA TTC G | [+20.0] | [+16.0] | [+7.0] | | |
| 5′-TCA TAT TCT ATA TCC C | 62.0 | 65.0 | 47 | 43.0 | +37.0 |
| 3′-AGT ATA AGA TAT AGG G | [+19.0] | [+22.0] | [+4.0] | | |
| 5′-CAC GGA ATT TAT ATG C | 63.0 | 65.0 | 59.0 | 50.0 | +19.0 |
| 3′-GTG CCT TAA ATA TAC G | [+13.0] | [+15.0] | [+9.0] | | |
| 5′-GTC TAT TAC AAT CCC | 59.0 | 60.0 | 49.0 | 46.0 | +24.0 |
| 3′-CAG ATA ATG TTA GGG | [+13.0] | [+14.0] | [+3.0] | | |
| 5′-GAT AAG TAG TAT TTC C | 61.0 | 61.0 | 42.0 | 43.0 | +37.0 |
| 3′-CTA TTC ATC ATA AAG G | [+18.0] | [+18.0] | [−1.0] | | |
| 5′-CTC CTA AGT ATG AC | 59.0 | 59.0 | 44.0 | 45.0 | +29.0 |
| 3′-GAG GAT TCA TAC TG | [+14.0] | [+14.0] | [−1.0] | | |

TABLE 6
Additional Bovine Sequences

Sequence
5′-Cy3 AGC CCT GTG 9 CCC TG
3′-TCG GGA CAC 9 GGG AC Cy3
5′-Cy3 AGC CCT GTG 4 CCC TG
3′-TCG GGA CAC 4 GGG AC Cy3
5′-Cy3 AGC CCT GTG N CCC TG
3′-TCG GGA CAC N GGG AC Cy3
5′-Cy3 AGC CCT GTG 9 CCC TG
3′-TCG GGA CAC GGG AC Cy3
5′-Cy3 AGC CCT GTG CCC TG
3′-TCG GGA CAC 9 GGG AC Cy3
5′-Cy3 AGC CCT GTG 4 CCC TG
3′-TCG GGA CAC GGG AC Cy3
5′-Cy3 AGC CCT GTG CCC TG
3′-TCG GGA CAC 4 GGG AC Cy3
5′-Cy3 AGC CCT GTG N CCC TG
3′-TCG GGA CAC GGG AC Cy3
5′-Cy3 AGC CCT GTG CCC TG
3′-TCG GGA CAC N GGG AC Cy3

Additional working embodiments of probes that may be used for gender determination in animals, more commonly, in bovine, are shown in Table 7, where Cy3 is a Cy3 fluorophore; underlined A/C/G/T are monomers; and underlined B is a bulged (non-pairing) monomer.

TABLE 7
Bovine Series Probe Target Region

| Probe | Target Region |
|---|---|
| 5′-AGC CCT GTG CCC TG | 5′-AGC CCT GTG CCC TG |
| 3′-TCG GGA CAC GGG AC | 3′-TCG GGA CAC GGG AC |
| 5′-CCT GTG CCC TG | 5′-CCT GTG CCC TG |
| 3′-GGA CAC GGG AC | 3′-GGA CAC GGG AC |
| 5′-CCT GTG CCC TG | 5′-CCT GTG CCC TG |
| 3′-GGA CAC GGG AC | 3′-GGA CAC GGG AC |
| 5′-AGC CCT GTG CCC TG | 5′-AGC CCT GTG CCC TG |
| 3′-TCG GGA CAC GGG AC | 3′-TCG GGA CAC GGG AC |
| 5′-CTG AAGC CCT GTG CCC TG | 5′-CTG AGC CCT GTG |
| | CCC TG |
| 3′-GAG TCG GGA CAC GGG AC | 3′-GAG TCG GGA CAC |
| | GGG AC |
| 5′-AGC CCT GTG CCC TG | 5′-AGC CCT GTG CCC TG |
| 3′-TCG GGA CAC GGG AC | 3′-TCG GGA CAC GGG AC |
| 5′-Cy3 AGC CCT GTG B CCC TG | 5′-AGC CCT GTG CCC TG |
| 3′-TCG GGA CAC B GGG AC Cy3 | 3′-TCG GGA CAC GGG AC |
| 5′-Cy3 AGC CCT GTG B CCC TG | 5′-AGC CCT GTG CCC TG |
| 3′-TCG GGA CAC GGG AC Cy3 | 3′-TCG GGA CAC GGG AC |
| 5′-Cy3 AGC CCT GTG CCC TG | 5′-AGC CCT GTG CCC TG |
| 3′-TCG GGA CAC B GGG AC Cy3 | 3′-TCG GGA CAC GGG AC |

The following Tables 8-10 describe the thermal denaturation properties of probes that may be used for gender determination of individual cells or multicellular assemblies from certain animals and humans; more commonly somatic cells, sperm cells or embryos from certain animals and humans; even more commonly, somatic cells, sperm cells or embryos from bovine.

As before, probes display thermostabilities that range from significantly lower to moderately higher than corresponding unmodified double-stranded DNA targets (note delta Tm values from −13 C to +9; column 4), while probe-target duplexes (column 2 and 3) are significantly more thermostable (range from +5 to +24 C). Accordingly, all of the probes (which have between two to five +1 zipper monomer arrangements) display significantly positive TA-values suggesting significant potential for targeting of double-stranded nucleic acid targets, more commonly dsDNA.

TABLE 8
Thermal Denaturation Properties of Exemplary Probes
Where T = 120Y; A = 120′W; C = 140′X and G = 140′Y

| | Upper | Lower | | | |
|---|---|---|---|---|---|
| | probe | probe | | | |
| | strand | strand | | | |
| | vs DNA | vs DNA | Probe | dsDNA | |
| | Tₘ[ΔTₘ] | Tₘ[ΔTₘ] | Tₘ[ΔTₘ] | target | TA |
| Probe | (° C.) | (° C.) | (° C.) | tₘ(° C.) | (° C.) |
| 5′-AGC CCT GTG CCC TG | 69.5 | 74.5 | 58.0 | 60.5 | +25.5 |
| 3′-TCG GGA CAC GGG AC | [+9.0] | [+14.0] | [−2.5] | | |
| 5′-CCT GTG CCC TG | 59.5 | 65.5 | 48.0 | 50.5 | +26.5 |
| 3′-GGA CAC GGG AC | [+9.0] | [+15.0] | [−2.5] | | |
| 5′-CCT GTG CCC TG | 59.0 | 64.0 | 47.0 | 50.5 | +25.5 |
| 3′-GGA CAC GGG AC | [+8.5] | [+13.5] | [−3.5] | | |
| 5′-AGC CCT GTG CCC TG | 69.5 | 75.5 | 61.5 | 60.5 | +23.0 |
| 3′-TCG GGA CAC GGG AC | [+9.0] | [+15.0] | [+1.0] | | |
| 5′-CTG AGC CCT GTG CCC TG | 74.0 | 78.0 | 57.0 | 66.0 | +29.0 |
| 3′-GAG TCG GGA CAC GGG AC | [+8.0] | [+12.0] | [−9.0] | | |
| 5′-AGC CCT GTG CCC TG | 70.0 | 80.0 | 60.0 | 60.5 | +29.5 |
| 3′-TCG GGA CAC GGG AC | [+9.5] | [+19.5] | [−0.5] | | |

Yet another working example of a particular embodiment is provided in Table 9 below, which shows thermal denaturation properties and TA-values for probes modified with unlocked monomer formula z.

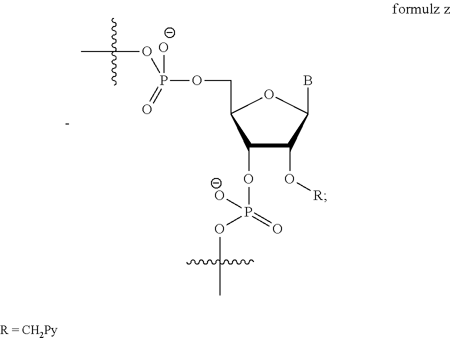

Similar patters as seen for other disclosed monomers are observed, i.e., probes display relatively low thermostability while probe-target duplexes are significantly more thermostable. Probes containing one or more +1 zipper arrangement of unlocked monomer formula z therefore display significantly positive TA-values and therefore significant potential for targeting of double-stranded nucleic acid targets, more commonly dsDNA targets.

TABLE 9
Thermal Denaturation Properties and TA-Values for Probes
Modified with Unlocked Monomer formula z

| | | | ‘upper’ | ‘lower’ | |
|---|---|---|---|---|---|
| | | | probe | probe | |
| | | | strand | strand | Target |
| | TA | Probe | vs DNA | vs DNA | DNA |
| Probe | (° C.) | Tm (° C.) | Tm (° C.) | Tm (° C.) | Tm (° C.) |
| 5′-GGformula z ATA TAT AGG C | 22.0 | 33.5 | 45.5 | 47.5 | 37.5 |
| 3′-CCA formula z AT ATA TCC G | | | | | |
| 5′-GGX formula z Aformula z A TAT AGG C | 25.0 | 43.5 | 51.5 | 54.5 | 37.5 |
| 3′-CCA formula z Aformula z ATA TCC G | | | | | |
| 5′-GGformula z Aformula z A formula z | 56.5 | 39.5 | 66.5 | 67 | 37.5 |
| Aformula z AGG C | | | | | |
| 3′-CCA formula z Aformula z Aformula z A | | | | | |
| formula z CC G | | | | | |

Particular embodiments entail double-stranded probes with certain zipper arrangements of monomers comprising so-called pseudo-complementary nucleobases (e.g., such as 2-thiouracil, 2,6- diamonopurines, inosine and pyrrolo-[2,3-d]-pyrimidine-2-(3H)-one), more commonly, +1 zipper arrangements of monomers comprising pseudo-complementary nucleobases, even more commonly, +1 zipper arrangements of monomers such as formula Y. Examples of working examples of these particular embodiments are given in Table 8 below.

Further particular embodiments entail double-stranded probes with certain zipper arrangements (more commonly +1 zippers) of monomers comprising nucleobases where, in addition, the nucleotide opposite of the disclosed monomer comprising a pseudo-complementary nucleobase is a nucleotide or disclosed monomer comprising a pseudo-complementary nucleobase (e.g., such a 2-thiouracil, 2,6-diamonopurines, inosine and pyrrolo-[2,3-d]-pyrimidine-2-(3H)-one). For a representative working examples, please see entries 2 and 4 in Table 29 below, where D is a DNA monomer with a 2,6-diaminopurine nucleobase (i.e., 2,6-diaminopurine-2′-deoxyriboside). With reference to Table 8 below, it observed that double-stranded probes with −1 or +1 zipper arrangements of monomer formula Y display positive TA-values, and therefore significant potential for targeting of double-stranded nucleic acid targets via the method disclosed in FIGS. 1-2, more commonly, dsDNA. With further reference to Table 8 below, it is observed that double-stranded probes with −1 or +1 zipper arrangements of monomer formula Y, where, in addition, the nucleotide opposite of monomer formula Y is D display positive TA-values, and therefore significant potential for targeting of double-stranded nucleic acid targets via the method disclosed in FIGS. 1-2, more commonly, dsDNA.

TABLE 10
Double-Stranded Probes with −1 Or +1 Zipper
Arrangements of Monomer formula Y

| | | Upper | Lower | | |
|---|---|---|---|---|---|
| | dsDNA | probe | probe | | |
| | target | strand | strand | Probe | |
| | Tₘ | vs DNA | vs DNA | Tₘ | |
| Probe | [° C.] | Tₘ [° C.] | Tₘ [° C.] | [° C.] | TA |
| 5′-GTG A(formula Y)A | 29.5 | 41.0 | 40.5 | 28.5 | +23.5 |
| TGC | | | | | |
| 5′-GTG A(formula Y)D | 29.5 | 45.0 | 45.0 | 29.5 | +31.0 |
| TGC | | | | | |
| 5′-GTG A(formula Y)A | 29.5 | 41.0 | 32.0 | 39.5 | +4.0 |
| TGC | | | | | |
| 5′-GTG D(formula Y)A | 29.5 | 42.5 | 33.0 | 29.0 | +17.0 |
| TGC | | | | | |

It is to be understood that the invention is not limited in its application to the details of construction and the arrangement of components set forth in the foregoing description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any nonclaimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for separating a population of sperm cells or embryos comprising:
a) connecting the population with a labeled locked nucleic acid capable of binding a gender-specific tandem repeat sequence in a portion of the population to provide a labeled fraction and an unlabeled fraction; and
b) separating the labeled fraction from the unlabeled fraction.

2. The method of claim 1, wherein the locked nucleic acid comprises an invader locked nucleic acid.

3. The method of claim 1, wherein a plurality of locked nucleic acids bind to the gender-specific tandem repeat sequence in step (a).

4. The method of claim 1, wherein the locked nucleic acid is labeled with a fluorescent tag, a heavy density tag, a magnetic tag, a nanoparticle, a toxin, a DNA, a siRNA, an enzyme, a specific ion, and combinations thereof.

5. The method of claim 4, wherein the population comprises sperm cells and at least 70% of the cells of the labeled fraction comprise a Y chromosome and at least 70% of the cells of the unlabeled fraction comprise an X chromosome.

6. The method of claim 4, wherein the population comprises sperm cells and at least 70% of cells of the labeled fraction comprise an X chromosome and at least 70% of the cells of the unlabeled fraction comprise a Y chromosome.

7. The method of claim 1, wherein the population comprises sperm cells, and wherein at least of the cells of the labeled fraction or the unlabeled fraction are viable after step (b).

8. The method of claim 1, wherein the population comprises sperm cells and wherein separating the cells in step (b) comprises physical separation by flow cytometry, centrifugation, magnetic force, or chemical separation using processes affecting metabolism, viability, motility, integrity or fertility.

9. The method of claim 1, further comprising permeabilizing the sperm cells or embryos prior to or during step (a).

10. The method of claim 9, wherein the sperm cells or embryos are permeabilized using electroporation, liposomes, osmotic pressure, or permeating peptides.

11. The method of claim 9, further comprising the use of micro, nano or other particles to facilitate penetration of the locked nucleic acid into the sperm cells or embryos prior to or during step (a).

12. The method of claim 9, wherein the locked nucleic acid penetrates the permeabilized sperm cells or embryos by electroporation, liposomes, nano or micro particles, osmotic pressure, or permeating peptides.

13. The method of claim 1, wherein the gender-specific tandem repeat sequence comprises a telomeric sequence.

14. The method of claim 1, wherein the gender-specific tandem repeat sequence is from about 2,000 to about 10,000 nucleotides.

15. The method of claim 1, wherein the labeled oligonucleotide is from about 12 to about 24 nucleotides.

16. The method of claim 1, wherein the population comprises mammalian sperm cells or mammalian embryos selected from bovine, porcine, canine, and equine.

17. A sperm cell or embryo comprising a gender-specific tandem repeat sequence and a locked nucleic acid bound to the gender-specific tandem repeat sequence.

18. The sperm cell or embryo of claim 17, wherein the locked nucleic acid is an invader locked nucleic acid.

19. The sperm cell or embryo claim 17, wherein the locked nucleic acid is labeled with a fluorescent tag, a heavy density tag, a magnetic tag, a nanoparticle, or combinations thereof.

20. A population of sperm cells, each cell in the population comprising an X chromosome comprising a gender-specific tandem repeat sequence or a Y chromosome comprising a gender-specific tandem repeat sequence, wherein at least 30% of the cells comprise a locked nucleic acid bound to the gender-specific sequence.

21. The cells of claim 20, wherein at least 70% of the cells comprise the locked nucleic acid bound to the gender-specific sequence.

22. The cells of claim 20, wherein at least 90% of the cells comprise the locked nucleic acid bound to the gender-specific sequence.

23. A method for identifying the sex of an embryo, the method comprising:
(a) contacting at least one cell of the embryo with a locked nucleic acid the locked nucleic acid comprising a label and capable of binding a gender-specific tandem repeat sequence, and
(b) detecting the presence or absence of the label in the embryo.

24. The method of claim 23, wherein the at least one cell of the embryo is viable.

25. The method of claim 23, wherein each cell of the embryo is contacted with the locked nucleic acid.

26. The method of claim 23, wherein the embryo comprises the locked nucleic acid bound to the gender-specific tandem repeat sequence and wherein the embryo is viable.

27. The method of claim 23, wherein the label comprises CY3 and wherein the label is detected using fluorometric techniques.


SEQUENCE LISTING (14 sequences)

SEQ ID NO: 1 - DNA, 3832 bp, Sus scrofa
gaattctcattaggtcccttatgtgtaggtggtggaaaagatgttgatctcggagtcaaatgtgtgatcatgtagaacgcagtggcaatgggatggttatctgtcagtgtgaagggctgagaatggtaaggtatgggtgcacggtgtaaggtgaaatatctccatacaagaagcggagttacttcctgcgcaaagaatttgtagttgataggagtttggaagactgatggagaatctcggttaaagaatttgactgctctcctggctgagatttgccaacctggaccagcacagataaccttggctgaggaggattcgagagaatgtgaaggaggaagaagccgagagagagagagagatgaaatttggatacaagccacattccagggcacagtttcattcccaagcccttgtctgtgtttccggggactattagacacgaaggctgtatcaggactcttgcatgagattttcactcgttcttggcctttctctagaagtacgtcgattgccagcagtccgtgcttggcacccatgtcttctcaaagtacacgaggtgtgggtcagttaaatgacaccctatattttccctaagcggcaaagacactgcctattgaaatggcagctcatagggtgtccttcctcagtcgttccttgatttcattgtccaggggcctttagtttgggtgtttggaatgatatcttttctctccaaaagagattctatactgtgtattcgtgtgaagcagtgactgagaaagcacttgaccctgaagtgctgcacatgttatcagatatccatcatgatagagaaaattgggctccccagtgtgtggagttgccttccttcggaaaggtgcagtcccagaggtggccatggttctgatatcatgcccatggagccttgtgcccaaacaacctctctgtgagaaaagaggtgtgtgatgggccatatgtgggaaagtctcaccctcaaaccttgagagttaaaggatggacaccctgagctcttgctctggaggtctgtcgaagttgatggaaaggaggatccatcagatgggtggtccttttggatgaacactattatcgccatctgtagactcatagccaccactctcatcaggctcccaagctcaatgtggattctagaggcttggtgtttgaacatgtgatatcttcaggggatccgtgggcagaatacctcacgggagttcctccatgtgaaaaaagagttgtgcttcagggccatttgaaacaatacctcccgcggcacaaagaaggaatccatgcccgaatgctctccaccaagcgtgaggccctggatgtaacccctcaaagagactttcccaatggggcattttcagctccacacttctgaatccatatttccatagcctaagccacaggggagccctccaaacagaggcctttaaaccaagatgtatccggtctatttcaggtgccttttccacaggtggataagaacaagccctcccactcattgagtgacttactgggagtgtggtgcctggggccagggccagtgtgtttgttctatctacagtcataaggaaggaagctgggacgatgatctgatcttccaggagcgctgtgatgatgcctgcaatgtgagggtattgaaagttccctcctggaagggagtgggctctgtccctgacactcctgaggctagagaatgaggaggtctaatccatagagactggtttttgttgacagacatcttgcactctgggctcttgttggccagcccactcgccaaggatgctcagaccattgtttggatgcccagtcattgagacatgaagagtcctagacatcatattctatatcccctaagaacgagtccagagatcctcacatgagcaaatccaccatacctcatggaccaggtagggaatgctgcagatcagcactttcttgggagagcactcatgggaacgttgtttcaagtgcgttgcactctgcgtccaatatgggaccctcgagaccgaagtacatatgaagtggtcagcgtgtccataggaagggatgactatgggaagtatggtttaaggctgacctagttggggtgtctccacacagcacaggaagtctttcaggaccacggaatttatatgcgatcagacctaggatgacagggagaaaaggctgggggataacttggacgattctcttggcaaatatatttgatccattattaccccaggtcgaattgtagcaggaggatacaggagaaattgaaggggggcaaaggggagagtgagagagctagagagaaggagatgaaaagaaggcgagagagaggtaaaggagctgttgtttcaagcaactgtcaatggcaggataatttctccaaggacgtgtctttctttaaggtggctagcacacaaatgggacaaaccagcacacttgtcccagagtcttctctccttcaaggcctgacgcccacattgtgtctattacaatccctgctccatcctggaagacaggtttgaaagccttgctgtggtccctctatcttctccaatggcatggggtggaaatcagagtaagagatattccatagtttccttcaggggaaaagcctcctcccatggaaagagatgctcgtgtgttgttcttcctccatcggccattgttttcctgttcaggctccgtggggagtgggtctttttcaatgccatattttctttcctaatgaggttgtagactgtgtattcctgggaagcagttttggaaccaccgtgttgagcctgacacacgttcaggggaagaggcatccttctgtagagagcaggtgggcacagaggaggtggagttgccttccatctagaagttcaggcccaatggttgccatggcaagggcatgttgcgcaccaagtattgtgacacaacacaccgtatgtgagaagagaggcgagggatgggcccaaagtgggaagctctcaccgtcacccccaattggttgtacctcgggcagcctgatctcatgatctggaggtatgttgatactaaatgaaacgaggatccattggatgtattggacaattggatgataagtagtatttccagcattcttctcatggccaccattctcatcaggctgctgaggaaataggcactgctcaggcttgcttttggatgatgatccatttgaaagggaccagtgagcctatcccttccagtcttttccaacagggaaaaaaagactcctaagtatgaccgggatgagaaatctcattccagatctcaaagcctgaaaattggccactcagccttccaaaaaagacgaggccctcaacatcaacccttacgaaatactggaatgggggcaaggaccattccttgcttcggaacgtacacgtccatagatgaaatcacaaactagctctccacagtaacaaatttcccaagaatgaagctgggatatgtctggtgggtatttcacaggttgataagaataagtcctgtctaaaggggtgctggatggaagtggggtgactagttccactgctggctgtttgtggggtacacagttgtcaggaatttaaggagtctctcttgttatcttccgtgagccctgtggtgagggctccccttgggcaggacttgaggtttcgctcttggacgagaggggcctctggcctgccaatggagggtctagagaaggaggaggattgtcccaaatggagactggtttgaggggaccgaatcctggaactctgtcccattgtcattcagaccctgctgaagggtgctcagaccccttcttagatgccaaatcactgagacatggagaggcagagaattc

SEQ ID NO: 2 - DNA, 14 bp, Sus scrofa
ctcctaagtatgac

SEQ ID NO: 3 - DNA, 313 bp, Sus scrofa
gaggaaagttgcactttcacggacgcagcctcccagatgggccctagctgggtccctccctacctgtagaaaggtgaggtggtggggccacttgccacacaaggcatattctggccccatccgcctcctccctgaagtagagcacgtttggagttggtttccagctctagcaatgacctgcaaagcaccagtgcacagggagcaggaggcagcccagaccctccttgttcctatggcgagcaatgggctaggggagaaaccagaaagcgctgctttcctgacgaaacacgcattgggctgagcttggtttccc

SEQ ID NO: 4 - DNA, 14 bp, Sus scrofa
tccgcctcctccct

SEQ ID NO: 5 - DNA, 1399 bp, Bos taurus
aattcaggctgcctcttgtgttggcccaggcaagtccaatcttccattcgagttgcgaaggaaagctggggattgctctcgagtgactgcagggccaatagacctcatctaggcttgtgtccagaagccaatgttcctctccaggggcgacagggatctcggggttgcattccagacgcacccggggagacaggcattcatctcgagtggaagcaaagaaccccgctctgctctcgaattgtgacgggtatctcttggagctcactgggtggactcaagggagtcaagcctcctgaggcgtttggagagaggtcgcgagattggtctctaggccatgcaggagacgaaggccctcatctctcgatgacggcccaatctcggggttgttctcgagcggcggccccagtgtgcggtttctcacgaggtacaacggcgaggtcagtgagcctctcgtggggcgccagggaagtcgggtctccatgcgagtggcgagggggagcgcgtcattgctcccgagccatggtaggggaatctggcctcgagacgtgttgaagaaggtctctcgagggctttcccgggttgaggcaggaaaccctgggttccctcgacttgtgcaggtgacctcagggggcttctcacggtggctctgagaagccagggaaactggaggtgggaggggcctcttgggactccactgggcttggtgcattggaagagggcctcatctccagtggaggcaggaaccgcaggtacctctgatttcagactccgatcgcagggtccctgcagactggggacaggagagtcaggcctcgtcttgggttgaggcatggaactccgcttgcctctcgagatgtccccggggagagaggccgcttgtcgagctgtatttggaacctggggttttttccgaacgatgcacggaaaaactgccccctcgtgttgacttcattcacaggctggagttcggagaggtgtccgggcatcgggttcttatcaagaggggaccgggaaatcggggtcctacggaatgtggaaccacccacgaggccacgtctggaatgtcttcgtgagaccggcctcatcctgaggtgcgaccggaaggtcgggaaccccttccagacaaagcaggggagtcgaccctcctgtccagatcaggaggggagaaagggctcagaggagggggtgccggaaaacctcagtgttcctctcgagggagaccgggatttcggggaactttgtgggtcgcatcaagggtgccaagtgccctttcgacctccaattcctaacgtgggacttctcctgaggcgctgtagccccaaagggcttcatcttgcgatgacgggggagccacgtggtttttctcgagttacggcgggattctcaagttgcgacgggg

SEQ ID NO: 6 - DNA, 16 bp, Bos taurus
cactattatcgccatc

SEQ ID NO: 7 - DNA, 14 bp, Sus scrofa
gaggattcatactg

SEQ ID NO: 8 - DNA, 14 bp, Sus scrofa
ctcctaagtatgac

SEQ ID NO: 9 - DNA, 14 bp, Sus scrofa
aggcggaggaggga

SEQ ID NO: 10 - DNA, 14 bp, Sus scrofa
tccgcctcctccct

SEQ ID NO: 11 - DNA, 16 bp, Bos taurus
gtgataatagcggtag

SEQ ID NO: 12 - DNA, 16 bp, Bos taurus
cactattatcgccatc

SEQ ID NO: 13 - DNA, 1184 bp, Bos taurus
atgcaagcccgggatctcagccctgtggtctgggaactgtgaaaccggcttgagtatgtgtgctgttatcagcactgtgccctggcgactctgatactggtttgtgttcatgtgtgtgtgtgtgtgtgtgtgtgttgctgttctcagccctgtgccctggcgattgtgcaaccagtatctgtatgcctgtgtgtgtgtgtgtgtgtgtgtgtgtgtgtgtgtgtgctgttctcaacccattgccctggcgattgttcaaccagtttgtgtatatgtgtgtgtagatgtgtgtgccatcctgagccttgtgccctggcaactggggaaacggtgtgtgtgtgttgtgtgtctgtgtgtgtgctgatttcagccatgtgcccttctgactgtgcaactggtttgtgtgtgtgtgcacgcgattctcacctctgtgtcctggcgactgtgtaaccgtttgtgtgtgtgagtgtgtgtaagtgtgtgctcttttcagccctgtttcctagagactgtggaaccggttggtgtgtgtgtgtgtctgtgtgtgtgtgtgccattctcagccctgtgccctggcgactgtgcaatattttgtcgtgtgtgtgtgtgtgtgtatttgtgtgtgcaattcacagccctgttccctggcgactgtgcaagcagattgttgcgtatgtttctgtgtgtgtgtgtgtgtgtgtgtgtgtgtatgtgctgttctcagccctgtgccctggcaactgtgaaaccggtttgtatgtgtgtgtgtgtttgtgtgtgccattcacagccctgtgccctggcgactgtgcaagcagtttgtgtgtgcatgtgtctgtgtgtgtatgtgtctgtgtgtgcatgtgtctgtgtgtgttatatgctgttctcagccctgtgccctggcgactgagaaaccggttgtgtgtgtgtgtgtgtgtgtgtgtgtgtgtgccagtttcagccctgtgccttggtactgtgcaagtggtttgtgtgtgtgtgtgtagtgtatatgtgtgtgtgtggtttgaccagttttcagccctgtgccttagtgactgtgtaactggtgtgtgtgtgtgtgtgtgtgtgtgtgctcttctcagccctgtgccctgttgactgtgcaagcggtttgtctgtgtatgtgagtgggtgctgttctcatgcctgtgcactgg

SEQ ID NO: 14 - DNA, 20 bp, Bos taurus
cagccctgtgccctggcgac